(19) United States
(12) Patent Application Publication
(10) Pub. No.: US 2024/0108820 A1
(43) Pub. Date: Apr. 4, 2024

(54) ATOMIZATION DEVICE AND METHOD OF PREDICTING ATOMIZATION TIME FOR THE SAME

(71) Applicant: HCMed Innovations Co., LTD., Taipei City (TW)

(72) Inventors: CHIEN-SHEN TSAI, New Taipei City (TW); SHIH-CHAO LUO, New Taipei City (TW); YUAN-MING HSU, New Taipei City (TW); CHUN-CHIA JUAN, New Taipei City (TW)

(21) Appl. No.: 18/474,176

(22) Filed: Sep. 25, 2023

Related U.S. Application Data
(60) Provisional application No. 63/409,859, filed on Sep. 26, 2022.

(30) Foreign Application Priority Data
Sep. 6, 2023 (CN) 202311140329.3

(51) Int. Cl. A61M 11/00; G16H 20/13

(52) U.S. Cl. CPC A61M 11/005; G16H 20/13; A61M 2202/0007; A61M 2205/3303; A61M 2210/1039; A61M 2230/005; A61M 2230/40

(57) ABSTRACT

An atomization device and a method of predicting atomization time for the same are provided. The atomization device includes a control module, an atomization module and a breathing sensing module. The method includes: configuring the breath sensing module to detect inhalations of a user using the atomization device, so as to generate initial breath data correspondingly; and configuring the control module to perform: comparing inhalation data of the initial breath data with a valid inhalation standard to obtain valid inhalation data and filter noise; statistically analyzing the valid inhalation data to generate a predicted value of inhalation time; calculating an atomization time according to the predicted value of the inhalation time; and generating a driving signal to drive the atomization module to perform atomization according to the atomization time.

### CROSS-REFERENCE TO RELATED PATENT APPLICATION

This application claims the benefit of priority to Patent Application No. 202311140329.3, filed on Sep. 6, 2023, in the People's Republic of China. The entire content of the above identified application is incorporated herein by reference.

This application claims the benefit of priority to the U.S. Provisional Patent Application No. 63/409,859, filed on Sep. 26, 2022, which application is incorporated herein by reference in its entirety.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a device and a method for the same, and more particularly to an atomization device and a method of predicting atomization time for the same.

### BACKGROUND OF THE DISCLOSURE

Drugs from existing atomization devices must be administered at the right time for effective dosing. In other words, the atomization device should atomize the drug when the user inhales so as to allow the user to breathe in aerosols generated therefrom.

To reduce imprecision of the atomization timing caused by differences in inhalation behaviors, most of the atomization devices trigger feedback signals to guide or prompt the user for required intensity, duration, or speed of inhaling. For example, a prompt message may be issued to instruct the user to inhale for more than 2 seconds.

However, certain users may not be able to breathe exactly as recommended due to illnesses or incapacitation. In this case, it is quite difficult to force the user to learn a specific breathing pattern for adapting to the configurations of the atomization device.

In addition, some of the existing atomization devices immediately determine a start or end time of drug administration according to characteristics relating pressure or changes in flow. However, such a method is prone to error as a result of case-specific changes in pressure or flow caused by mis-operation, such as to falsely determine a dosing duration.

Therefore, it is necessary to design a method that can effectively predict a user's breathing time, reduce an inconvenience for users with limited capabilities, and filter out abnormal breathing patterns or signals.

### SUMMARY OF THE DISCLOSURE

In response to the above-referenced technical inadequacies, the present disclosure provides an atomization device and a method for predicting an atomization time, which can effectively predict a breathing time of the user, reduce an inconvenience caused by a limitation of the user's breathing capability, and has a mechanism for avoiding false triggering and filtering of multiple invalid triggers to filter abnormal breathing patterns or signals.

In order to solve the above-mentioned problems, one of the technical aspects adopted by the present disclosure is to provide a method of predicting atomization time adapted to an atomization device. The atomization device includes a control module, an atomization module and a breathing sensing module. The method includes: configuring the breath sensing module to detect one or more inhalations while a user is using the atomization device, so as to generate one or more records of initial breath data correspondingly; and configuring the control module to: compare the one or more records of inhalation data in the records of initial breath data with a valid inhalation standard to obtain one or more records of valid inhalation data; statistical analyze the one or more records of the valid inhalation data to generate a predicted value of inhalation time; calculate an atomization time according to the predicted value of the inhalation time; and generate a driving signal to drive the atomization module to perform atomization according to the atomization time.

In order to solve the above-mentioned problems, another one of the technical aspects adopted by the present disclosure is to provide an atomization device, which includes an atomization module, a breath sensing module configured to detect one or more inhalations when a user uses the atomizing device, so as to generate one or more records of initial breath data correspondingly, and a control module electrically connected to the atomization module and the breath sensing module. The control module is configured to: compare the one or more records of inhalation data in the records of initial breath data with a valid inhalation standard to obtain one or more records of valid inhalation data; statistically analyze the one or more records of the valid inhalation data to generate a predicted value of inhalation time; calculate an atomization time according to the predicted value of the inhalation time; and generate a driving signal to drive the atomization module to atomize a to-be-atomized medicine according to the atomization time.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a functional block diagram of an atomization device according to one embodiment of the present disclosure;

FIG. 2 shows a schematic view of a user using the atomization device according to one embodiment of the present disclosure;

FIG. 3 is a flowchart of a method for predicting atomization time according to one embodiment of the present disclosure;

FIG. 4 is a flowchart of an anti-mistouch mechanism according to one embodiment of the present disclosure;

FIG. 5 is a graph showing a pressure corresponding to the user's breathing in relation to time when the atomization device is used according to one embodiment of the present disclosure;

FIG. 6 is a detailed flowchart of step S31; and

FIGS. 7 to 11 are respectively schematic diagrams of first to fifth aspects of filtering multiple triggered signals according to one embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

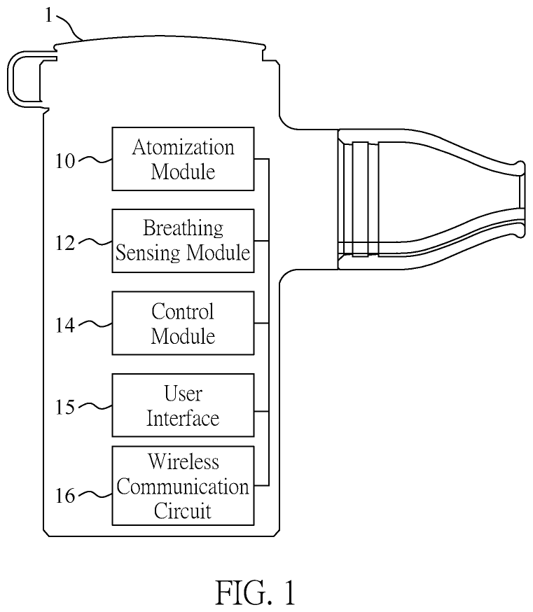

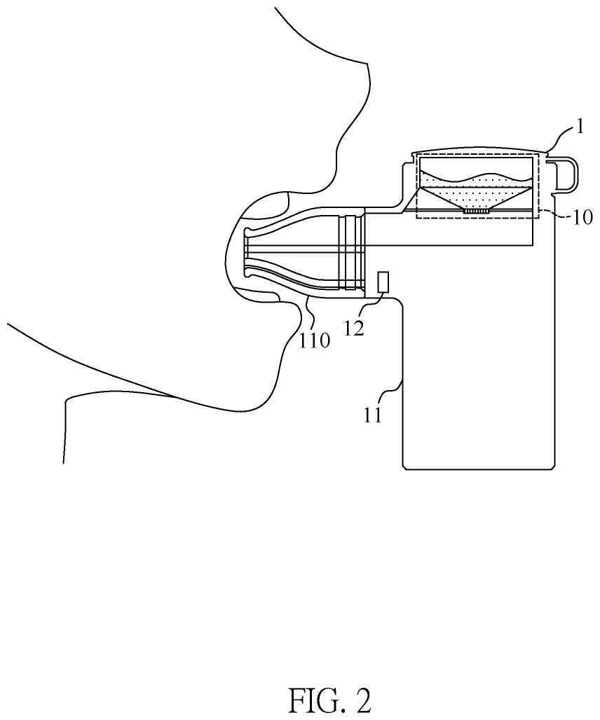

FIG. 1 is a functional block diagram of an atomization device according to one embodiment of the present disclosure, and FIG. 2 shows a schematic view of a user using the atomization device according to one embodiment of the present disclosure. Referring to FIG. 1, one embodiment of the present disclosure provides an atomization device 1 capable of accurately predicting an atomization time based on a user's inhalation time. The atomization device 1 includes an atomization module 10, a breathing sensing module 12, a control module 14, a user interface 15 and a wireless communication circuit 16.

The atomization module 10 can be, for example, an ultrasonic atomization module, a compression atomization module or a mesh atomization module. The present disclosure does not limit a type of the atomization module 10. Taking the atomization module 10 in FIG. 2 as an example, the atomization module 10 can include an atomization unit and a liquid container. The atomization unit (for example, a metal microporous sheet a mesh) can convert a medicinal liquid in the liquid container into aerosol with tiny droplets through a vibration generated by a powered piezoelectric element.

It should be noted that, as shown in FIG. 2, the atomization device 1 can include a host housing 11 having an air suction port 110 extending therefrom. The air suction port 110 includes two parts that are independent (pipe isolated) of each other, so as to avoid a deterioration of the breathing sensing module 12 due to adhesion of aerosols. One part of the suction port 110 (for example, an upper part) can be used for the user to inhale atomized medication after the atomization module 10 atomizes the medicinal liquid in the liquid container, and another part of the suction port 110 (for example, a lower part) allows the breathing sensing module 12 disposed in the host housing 11 to sense pressure and/or flow changes generated when the user breathes.

For example, the breathing sensing module 12 can include a pressure sensor and/or a flow sensor. When a user uses the atomization device 1, a pressure sensor and/or a flow sensor included in the breathing sensing module 12 can detect one or more breaths of the user, and correspondingly generate one or more initial breath data. Each record of the initial breath data can include pressure data, flow data and/or a duration detected by the breathing sensing module 12 during one breath of the user.

The control module 14 is electrically connected to the atomization module 10 and the breathing sensing module 12. The control module 14 can be, for example, a processor, a control chip or a microcontroller chip. The control module 14 can receive signals from the pressure sensor and/or flow sensor included in the breathing sensing module 12, and record the aforementioned initial breath data based on the signals. The control module 14 can also generate a driving signal for controlling the atomization module 10 to determine when the atomization module 10 performs atomization.

In addition, the control module 14 can also have a built-in memory for storing the above-mentioned initial breathing data and a plurality of computer-readable instructions. The control module 14 can be configured to execute the computer-readable instructions in the memory to implement the steps mentioned below.

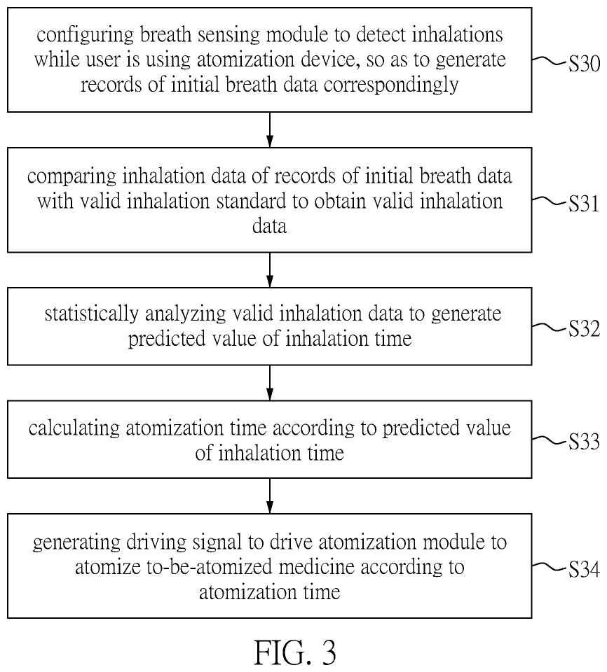

The embodiment of the present disclosure also provides a method for predicting the atomization time, which is suitable for the atomization device 1 shown in FIGS. 1 and 2, but the present disclosure is not limited thereto. FIG. 3 is a flowchart of a method for predicting atomization time according to one embodiment of the present disclosure. Reference is made to FIG. 3. The method for predicting the atomization time can include the following steps:

Step S30: configuring the breath sensing module to detect inhalations while a user is using the atomization device, so as to generate records of initial breath data correspondingly.

After step S30, the control module 14 is configured to perform the following steps:

Step S31: comparing inhalation data of the records of initial breath data with a valid inhalation standard to obtain valid inhalation data.

In step S31, the valid inhalation standard is set to determine whether or not each record of the inhalation data is a valid inhalation, so as to exclude abnormal breathing behaviors (such as coughing). The valid inhalation is defined by having a duration longer than a time threshold, having a pressure value measured by the pressure sensor to be lower than a pressure threshold (negative pressure, the lower the pressure value, the greater the pressure), and/or having a flow value measured by the flow sensor be higher than a flow threshold. If any of the above conditions is met, the inhalation can be determined as a valid inhalation.

Therefore, in step S31, a determination process can be executed for each record of the inhalation data. Each record of the inhalation data is determined to be one record of the valid inhalation data when the corresponding pressure data is lower than the pressure threshold or the corresponding flow data is higher than the flow threshold, and the corresponding duration is greater than the time threshold.

It should be noted that the pressure threshold and the flow threshold are parameters that can be adjusted according to user types, that is, can be set for different user groups. For example, a pressure noise detected by the breathing sensing module 12 is approximately ±1.3 Pa. A safety margin can be added based on such the pressure noise, and the pressure threshold can be set to approximately twice the pressure noise, that is, −2.55 Pa. In addition, the flow threshold can be a flow rate that causes the aforementioned pressure noise under the same design, that is, 8 L/min. According to different usage requirements and the design of the atomization device 1, the pressure threshold can range from 0 Pa to −15 Pa, and the flow threshold can range from 0 L/min to 100 L/min.

Similarly, the time threshold is also an adjustable parameter, that is, the time threshold can be set for different user groups, such as adults, children, infants, asthma patients, patients with obstructive lung diseases, restrictive lung diseases, infectious lung diseases, with interstitial lung diseases, vascular lung diseases, and/or lung cancer (e.g., respiratory tumors). Specifically, different patient groups have different lung capabilities and different inhalation intensity and length. In order to avoid misjudgment of the inhalation behaviors, the time threshold used can be adjusted if necessary. According to measurement, the breathing noise caused by environmental differences or incorrect use is within about 0.25 seconds, and most of inhalations having a duration that exceeds 0.25 seconds are normal inhalations. Therefore, the time threshold can be set to be greater than or equal to 0.25 seconds. That is, when the duration of the inhalation is more than 0.25 seconds, if the pressure data of the inhalation is lower than the pressure threshold, or if the flow data is higher than the flow threshold, the inhalation will be regarded as a valid inhalation. In addition to being adjusted according to different user groups, the time threshold can also be adjusted according to different drug properties, drug amounts, manufacturer requirements (for specific drugs), or other purposes.

Step S32: statistically analyzing the one or more records of the valid inhalation data to generate a predicted value of inhalation time.

In this step, last N records of the valid inhalation data can be retrieved from the obtained valid inhalation data as a basis for generating a prediction value of the inhalation time, and N is a positive integer greater than or equal to 2. For example, if N is set to 3, three durations corresponding to the last three records of the valid inhalation data can be retrieved and used to calculate and obtain the prediction value of the inhalation time. The calculation can be made by, for example, calculating an average (i.e., a moving average) of the records of the durations as the predicted value of the inhalation time.

It should be noted that in step S32, a quantity of the records of the valid inhalation data may be insufficient. Specifically, when the user inhales for the first time after turning on the atomization device 1, the quantity of the records of the valid inhalation data is zero, which is less than N (for example, 3), and there are three missing durations (obtained by subtracting the quantity of records (zero) from N). The three missing durations are all replaced by a time preset value for calculating the predicted value of the inhalation time, and the aforementioned moving average will be used.

Similarly, when the user takes a second inhalation and the first inhalation is determined as a valid inhalation, the quantity of the records of the valid inhalation data is one, which is less than N (e.g., 3), and there are two missing durations obtained by subtracting the quantity of the records (one stroke) from N. The durations of these two missing records are replaced by the time preset value for calculating the prediction value of the inhalation time (for example, moving average).

In this embodiment, the preset time value can also be set for different user groups. Hereinafter, a period of time within which the atomization device 1 has not obtained the user's actual inhalation time length and cannot accurately perform statistics and prediction is referred to as an initial dosing period. N inhalations of 1 second can be preset as a time preset value in order to avoid using excessive dosing time during an inhalation cycle of the initial dosing period, which may cause the drug atomized by the atomization device 1 at the end of the inhalation cycle to not be effectively inhaled by the patient, resulting in wastage of the drug and contamination. Under this condition, the predicted value (moving average) of the inhalation time calculated based on the N time preset values is 1 second, which is shorter than known inhalation lengths of most demographical groups, so as to avoid wastage of the drug and contamination. After obtaining a sufficient quantity of the valid inhalation data, the moving average can be calculated to serve as the prediction value, so as to gradually approach the user's true inhalation length.

In some embodiments, if the atomization device 1 is turned off and then started again, one or more records of the valid inhalation data before the shutdown can be used to calculate the predicted value of the inhalation time, and the present disclosure does not limit the quantity of the valid inhalation data to be used after the atomization device 1 is restarted. In some embodiments, if the atomization device 1 is turned off and restarted, the valid inhalation data before the shutdown is no longer used to calculate the predicted value of the inhalation time. On the contrary, the atomization device 1 is initialized to a condition that the quantity of records of the valid inhalation data is zero when the user performs a first inhalation, and the N time preset values are used as the basis for calculation. In this way, if the atomization device 1 is restarted and used by a different user or in a discontinuous use status that put a large difference in the inhalation time, it is possible to prevent the predicted value from deviating from actual situation and wasting drugs.

Step S33: calculating an atomization time according to the predicted value of the inhalation time.

In step S33, the atomization time can be a predetermined ratio multiplied by the predicted value of the inhalation time. For example, the predetermined ratio can be optimized for different groups of users (or different nature or amount of drugs), or customer needs (when the drug is bound for sale), or can be set according to other purposes. The predetermined ratio can be within a range of 1% to 100%, and can preferably be, for example, 70%. In some embodiments, the predetermined ratio can be set directly on the atomization device 1, for example, through the user interface 15 having buttons, displays and/or input devices. Alternatively, configuration information can be transmitted to the atomization device 1 through the built-in wireless communication circuit 16 for setting the atomization device 1.

In some embodiments, through the wireless communication circuit 16, the user's breathing parameters and related records (including visual patterns) can be transmitted to portable devices (such as mobile phones, tablet computers) for presentation, which can display in real time and be played back for reference in the future. The portable devices can execute an application program corresponding to the atomization device 1 to highlight specific breathing characteristics (e.g., overly short breathing durations, disorderly breathing) through data filtering or other data integration methods, so as to prompt the user that they need to adjust their breathing, rather than simply displaying breathing information. Furthermore, the portable device can limit data received from the atomization device 1 by graphical control or voice control.

Furthermore, in order to avoid wastage of medicine caused by misjudgment, or excessively long inhalation signals due to misuse of the device, the atomization time will not exceed a preset reasonable upper limit of dosing time. That is, the upper limit of the dosing time is not affected by a result obtained through multiplying the predicted value of the inhalation time by the predetermined ratio. For example, the upper limit can be set to 8 seconds, even if the predicted value of the inhalation time multiplied by the predetermined ratio is greater than 8 seconds, the atomization time can only be 8 seconds at most.

Step S34: generating a driving signal to drive the atomization module to atomize a to-be-atomized medicine according to the atomization time.

It should be noted that every time the user inhales, a starting point of the atomization time (i.e., a starting point of the dosing time) is at the time when the pressure value measured by the pressure sensor is lower than the pressure threshold and/or the flow value measured by the flow sensor exceeds the flow threshold.

During the breathing prediction, a corresponding feedback mechanism can be given to the user according to an operation status. For example, when the user inhales and triggers the atomization successfully, the atomization device 1 can vibrate once at a frequency of 1 second, and when a medicinal liquid is used up, the atomization device 1 can vibrate twice at a frequency of 1 second, but the present disclosure is not limited thereto.

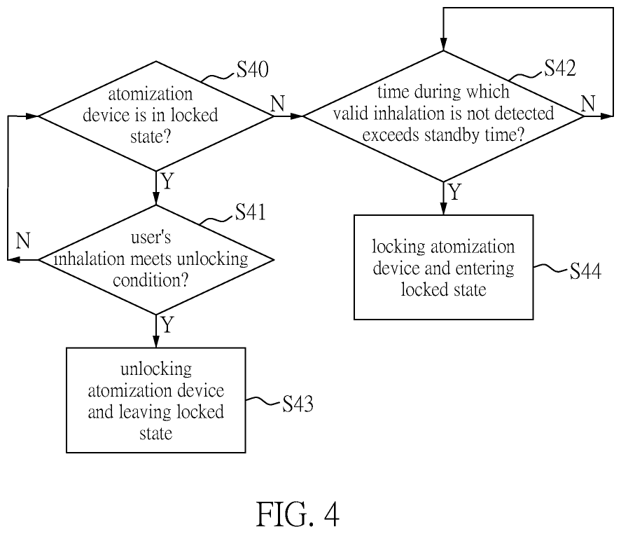

FIG. 4 is a flowchart of an anti-mistouch mechanism according to one embodiment of the present disclosure. In the flowchart of FIG. 4, in order to avoid setting the pressure/flow threshold too low, causing the atomization device 1 to be driven without the user inhaling, the present disclosure further provides an anti-mistouch mechanism, which includes configuring the control module to perform the following steps:

Step S40: determining whether or not the atomization device is in a locked state.

If affirmative, the anti-mistouch mechanism proceeds to step S41; if negative, the anti-mistouch mechanism proceeds to step S42.

Step S41: determining whether or not a user's inhalation meets an unlocking condition.

In step S41, the unlocking condition can be the same or stricter than the definition of the valid inhalation that is used to predict the inhalation time. For example, whether or not the pressure is lower than or equal to −5 Pa, whether or not the flow rate is higher than or equal to 15 L/min, or whether or not the duration is greater than or equal to 0.5 seconds, are determined in step S41.

In response to determining that the user's inhalation meets the unlocking condition, the anti-mistouch mechanism proceeds to step S43 to unlock the atomization device 1 and leave the locked state. In response to determining that the user's inhalation does not meet the unlocking condition, the anti-mistouch mechanism proceeds back to step S40 to again determine whether or not the atomization device 1 is in the locked state.

Step S42: determining whether or not a time during which valid inhalation is not detected exceeds a standby time.

In step S42, the standby time can be set to, for example, 8 seconds. When a valid inhalation is not detected for more than the standby time, the anti-mistouch mechanism proceeds to step S44 to lock the atomization device 1 and enter the locked state. If negative, step S42 is repeated.

It should be noted that when the atomization device 1 is in the locked state, the atomization module 10 will be disabled and cannot perform atomization. On the contrary, if the atomization device 1 leaves the locked state, the atomization module can be driven at any time according to the obtained atomization time.

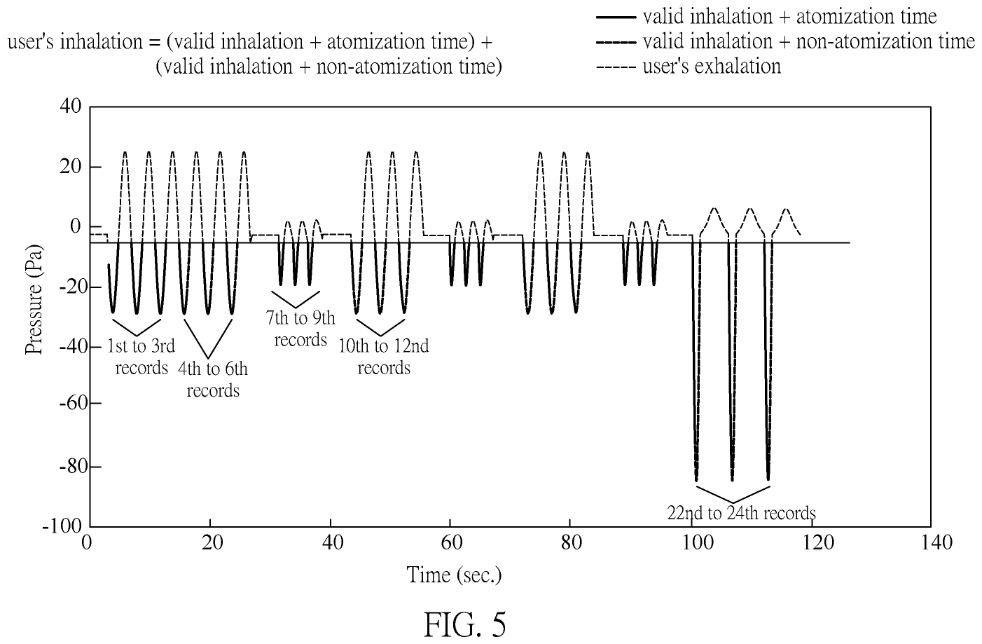

Reference is made to FIG. 5, which is a graph showing a pressure corresponding to the user's breathing versus time when the user uses the atomization device according to one embodiment of the present disclosure. In FIG. 5, the way by which the atomization device 1 detects the user's breath and performs atomization are shown.

The first three records of valid inhalations illustrate a case where the quantity of the records of the valid inhalation data is less than N (e.g., 3), and the atomization time has not been adaptively adjusted according to the durations of the user's valid inhalations. During the fourth to sixth records of valid inhalations, the drug can be administered stably with 70% of the durations as the atomization time. During the 7th to 9th records of valid inhalations, due to the sudden shortening of the inhalation time, the administration according to 70% of the predicted value of the inhalation time will occupy all the durations of the 7th to 9th valid inhalations. During the 10th to 12th and 22nd to 24th records of valid inhalations, it can be seen that the atomization device 1 has adapted to the shorter duration of the valid inhalations, and after averaging several times, the atomization time will slowly approach the user's actual durations of the valid inhalations.

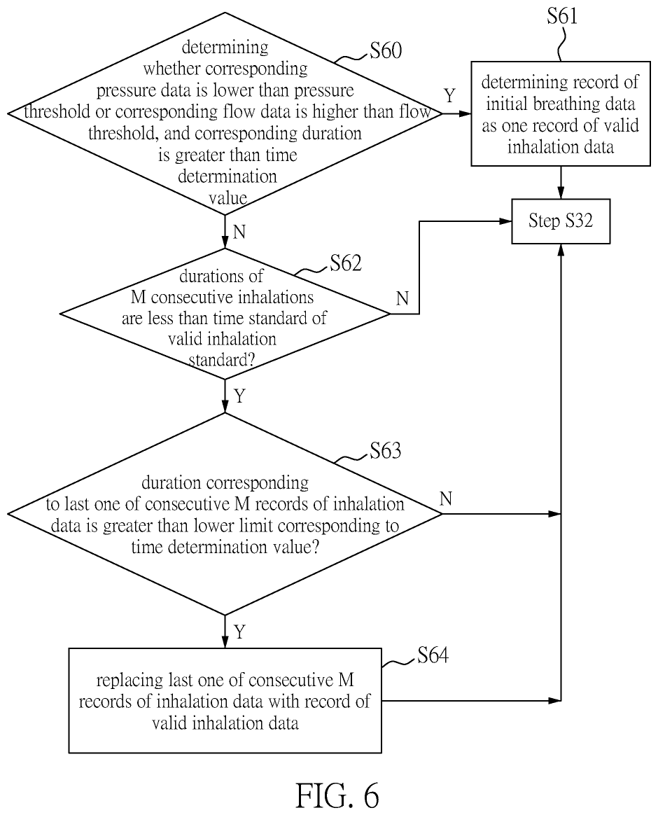

It should be noted that in step S31, the valid inhalation can be defined in other ways. Reference is made to FIG. 6, which is a detailed flowchart of step S31. As shown in FIG. 6, step S31 can include the following steps:

Step S60: for a record of the initial breathing data, determining whether or not corresponding pressure data is lower than the pressure threshold or corresponding flow data is higher than the flow threshold, and a corresponding duration is greater than a time determination value.

If affirmative, step S31 proceeds to step S61: determining the record of the initial breathing data as one record of valid inhalation data. Step S31 proceeds to step S32.

It should be noted that the time determination value different from the aforementioned time threshold is further introduced in the present embodiment. Similar to the time threshold, the time determination value can be obtained by multiplying the prediction value of the inhalation time by a user ratio (for example, an adaptability ratio) related to the user, and the time determination value is also a parameter that can be adjusted for different user groups. Specifically, the time determination value can be a range, for example, a determination parameter (expressed as %) greater than or equal to the prediction value of the inhalation time with additional lower and upper limits.

For example, if the initial prediction value of the inhalation time is 1 second, and if the determination parameter is set to 25% according to the time threshold of 0.25 seconds (equivalent to 25% of an initial inhalation time of 1 second) of the aforementioned embodiment, then the time determination value can be calculated as: 1×0.25=0.25 seconds. After multiple valid inhalations, the calculated prediction value of the inhalation time increases to 2 seconds. According to the determination parameter of 25%, the time determination value can be calculated as: 2×0.25=0.5 seconds. That is, every time when the user inhales, the inhalation will be regarded as a valid inhalation to be included in the calculation of the prediction value of the inhalation time only if a pressure value measured is lower than the pressure threshold for more than 0.5 seconds. In other words, a condition for determining the valid inhalation is not set to a fixed value as the time threshold, but can be adjusted accordingly according to changes in the predicted value of the inhalation time. In this way, a criterion for determining the valid inhalation can be adjusted according to the user's breathing capability.

In the present disclosure, the determination parameter is not limited to 25% mentioned above, and can be adjusted according to different user groups (or drug properties, drug dosage), or customer needs (when binding drugs for sale), or other purposes. For example, if the inhalation time of patients with obstructive pulmonary disease is approximately half that of normal adults, the determination parameter can be reduced to 10 to 15% to avoid determining inhalations that reach the patient's limit as invalid inhalations, or determining actual inhalations as noises.

In addition, in order to prevent the time determination value from being excessively lowered or raised due to an influence of extreme values, upper and lower limits can be set for the determination value. For example, the lower limit is 0.25 seconds and the upper limit is 2 seconds, and the upper and lower limits are not affected by the calculated time determination value. If the inhalation time is gradually shortened and less than 1 second, for example, 0.8 seconds, the time determination value is calculated as 0.8×0.25=0.2 seconds, and if 0.2 seconds is lower than the lower limit of 0.25 seconds, the lower limit of 0.25 seconds is used as the time determination value, not 0.2 seconds. On the other hand, the time determination value is also determined by the upper value. The reason for using 0.25 seconds as the lower limit is that, according to measurement, the breathing noise caused by environmental differences or incorrect use is within about 0.25 seconds, and most of inhalations having a duration that exceeds 0.25 seconds are normal inhalations. Taking 2 seconds as the upper limit is because most normal people will not inhale for more than 8 seconds, and 25% of 8 seconds is 2 seconds.

In response to determining that the inhalation is not the valid inhalation in step S60, step S31 proceeds to step S62: determining whether or not durations of M consecutive inhalations are less than the time standard of the valid inhalation standard. If negative, step S31 ends and the method proceeds to step S32.

In step S62, when adjusting the standard for determining the valid inhalation according to the user's breathing capability, an actual inhalation duration may suddenly drop and be stabilized at a short inhalation time that continues to be less than the calculated time determination value and causing the moving average to be unable to be correctly adjusted to be the same as the actual inhalation duration. To avoid such a situation, the time determination value can be set as not being applied continuously for more than M times. M is an integer greater than or equal to 2, which can be, for example, 3.

Therefore, when the duration corresponding to the inhalation data is less than the time standard of the valid inhalation standard (i.e., the time determination value) for M consecutive times, the method proceeds to step S63: determining whether or not the duration corresponding to a last one of consecutive M records of the inhalation data is greater than a lower limit corresponding to the time determination value.

If affirmative, the method then proceeds to step S64: replacing the last one of the consecutive M records of the inhalation data with a record of the valid inhalation data (which is the lower limit corresponding to the time determination value). The method then proceeds to step S32. If negative, step S31 ends and the method proceeds to step S32.

It should be noted that in the embodiment using the time determination value, the predicted value of the inhalation time is generated in step S32, and the time determination value needs to be updated synchronously, so that when the initial breath data increases, step S60 is executed again, and the initial breath data is generated again according to the new valid value. The new valid inhalation determination criteria are used to determine whether the latest initial breath data is valid inhalation.

However, if an excessively irregular short-breathing signal is generated due to user or environmental factors, it may cause the atomization module 10 to be erroneously driven. Therefore, the present disclosure further establishes a mechanism for filtering multiple invalid triggers to prevent the atomization module 10 from being erroneously driven.

Specifically, the method for predicting the atomization time of the present disclosure is further implemented according to a filtering standard. The filtering standard has a lock time threshold for locking and an unlock time threshold for unlocking. “Locking” refers to activating an invalidation of a function of the control module 14 sending a drive signal to drive the atomization module 10, and “unlocking” refers to deactivating the invalidation of the function of the control module 14 sending the drive signal when a valid inhalation is detected under the “locked” condition.

The condition for activating “locking” is that, after one or more records of the inhalation data are determined to be valid inhalation data, if a duration corresponding to the user's current inhalation is greater than the locking time threshold, when another valid inhalation (hereinafter referred to as a second valid inhalation) is detected again after the end of the previous valid inhalation, the function of the control module 14 corresponding to the second valid inhalation to drive the atomization module 10 (such as a microporous sheet) is deactivated. That is, if the duration corresponding to the user's current inhalation is greater than the locking time threshold, the driving signal sent by the control module 14 will not be able to drive the atomization module 10 during the next valid inhalation.

When locked, a condition for unlocking is that, when a continuous accumulated time of one or more invalid inhalation signals after the user stops the operation (i.e.: no inhalation or exhalation is detected) is greater than the unlocking time threshold, the invalidation of the function of the control module 14 drives the atomization module 10 is deactivated when a next time a valid inhalation is detected. That is, when “locking” is activated, if the continuous accumulated time of the one or more invalid inhalation signals after the user stops inhaling is greater than the unlocking time threshold, the unlocking can be performed, such that the driving signal sent by the control module 14 can drive the atomization module 10 normally when the next valid inhalation is detected.

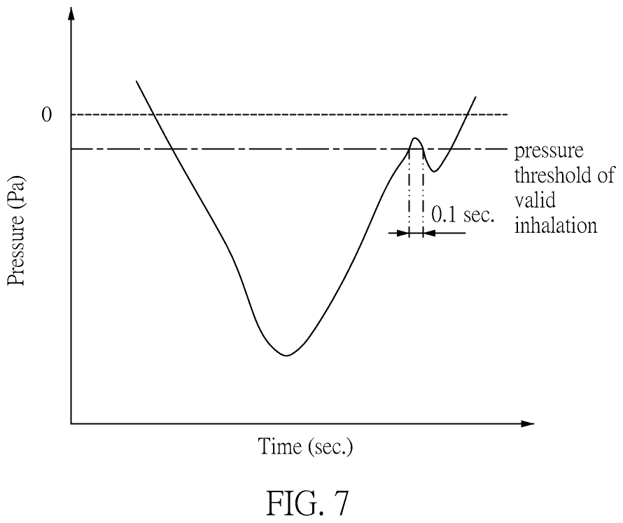

Reference is made to FIGS. 7 to 11, which are respectively schematic diagrams of first to fifth aspects of filtering multiple triggered signals according to one embodiment of the present disclosure. For example, when the locking time threshold is set as 0.25 seconds and the unlocking time threshold is set as 0.3 seconds, as shown in the first aspect of FIG. 7, based on a premise that the user's first inhalation is determined as a valid inhalation, since a duration of the user's inhalation (i.e., a period of time during which a pressure is measured to be lower than the pressure threshold) is greater than 0.25 seconds, the function of the control module 14 driving the atomization module will be invalidated next time a valid inhalation is detected. Subsequently, since a stop time (i.e., a period of time during which the pressure measured to be greater the pressure threshold) after the user stops inhaling (such as no inhalation or exhalation) is 0.1 seconds, which is less than 0.3 seconds, when the user is in the second inhalation, the control module 14 will not be unlocked, such that the atomization module 10 will not atomize the medicinal liquid even if the user is in the second inhalation.

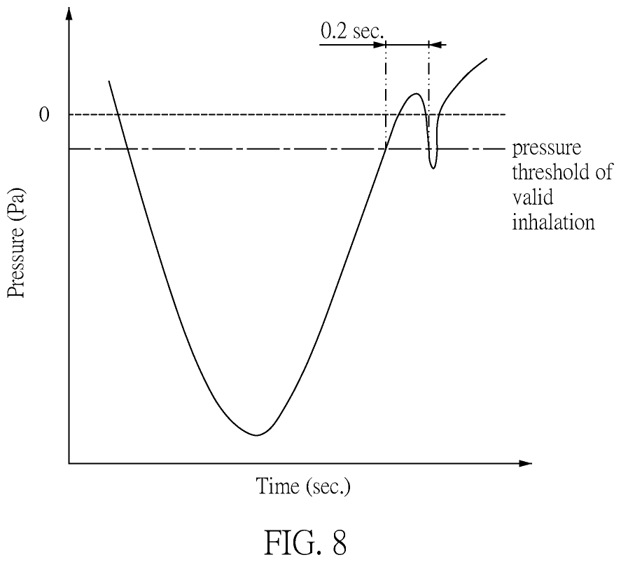

As shown in the second aspect of FIG. 8, based on a premise that the user's first inhalation is determined as a valid inhalation, since a duration of the user's inhalation (i.e., a period of time during which a pressure is measured to be lower than the pressure threshold) is greater than 0.25 seconds, the function of the control module 14 driving the atomization module will be invalidated next time a valid inhalation is detected. Subsequently, since a stop time (i.e., a period of time during which the pressure measured to be greater the pressure threshold) after the user stops inhaling (such as no inhalation or exhalation) is 0.2 seconds, which is less than 0.3 seconds, when the user is in the second inhalation, the control module 14 will not be unlocked, such that the atomization module 10 will not atomize the medicinal liquid even if the user is in the second inhalation.

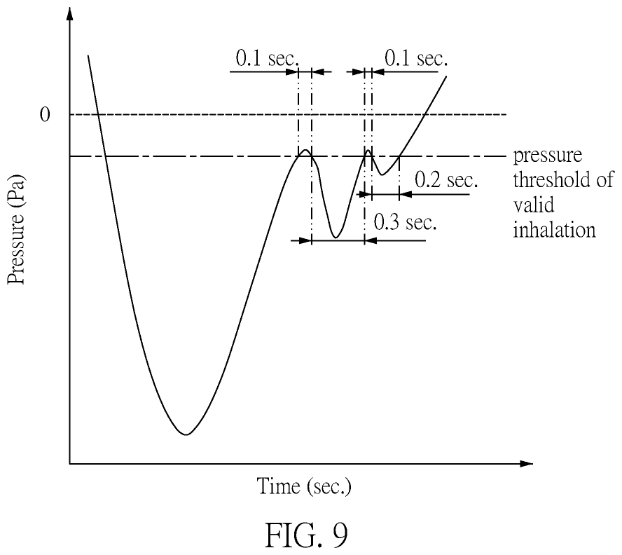

As shown in the third aspect of FIG. 9, based on a premise that the user's first inhalation is determined as a valid inhalation, since a duration of the user's inhalation (i.e., a period of time during which a pressure is measured to be lower than the pressure threshold) is greater than 0.25 seconds, the function of the control module 14 driving the atomization module will be invalidated when next time a valid inhalation is detected. Subsequently, since a stop time (i.e., a period of time during which the pressure measured to be greater the pressure threshold) after the user stops inhaling (such as no inhalation or exhalation) is 0.1 seconds, which is less than 0.3 seconds, when the user is in the second inhalation, the control module 14 will not be unlocked, and the atomization module 10 will not atomize the medicinal liquid. Finally, since a stop time (i.e., a period of time during which the pressure measured to be greater the pressure threshold) after the user stops inhaling for the second time is 0.1 seconds, which is less than 0.3 seconds, when the user is in a third inhalation, the function of the control module 14 driving the atomization module 10 remains invalidated, such that the atomization module 10 will not atomize the medicinal liquid even if the user is in the third inhalation.

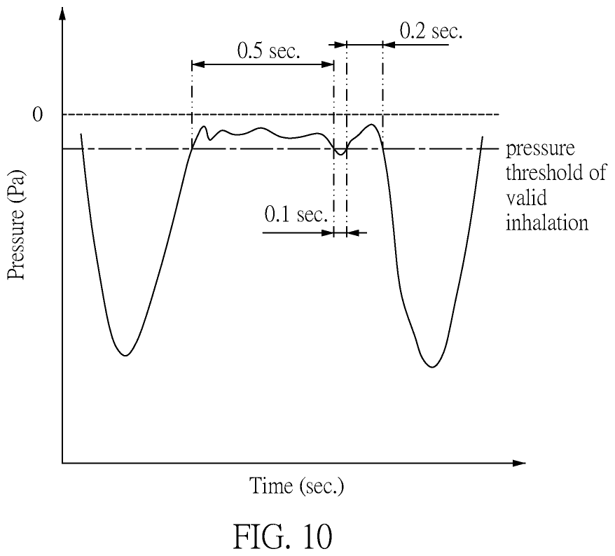

As shown in the fourth aspect of FIG. 10, based on a premise that the user's first inhalation is determined as a valid inhalation, since a duration of the user's inhalation (i.e., a period of time during which a pressure is measured to be lower than the pressure threshold) is greater than 0.25 seconds, the function of the control module 14 driving the atomization module will be invalidated when next time a valid inhalation is detected. Subsequently, since a stop time (i.e., a period of time during which the pressure measured to be greater the pressure threshold) after the user stops inhaling is 0.5 seconds, which is greater than 0.3 seconds, when the user enters a second inhalation, the invalidation of the function of the control module 14 driving the atomization module 10 will be deactivated, then the atomization module 10 performs atomization and dosing. Then, since a duration of the user's second inhalation is 0.1 seconds, which is less than 0.25 seconds, the function of the control module 14 driving the atomization module 10 will not be invalidated. Therefore, even if a second stop time is 0.2 seconds, which is less than 0.3 seconds, the atomization module 10 will still atomize the medicinal liquid when the user is in the third inhalation.

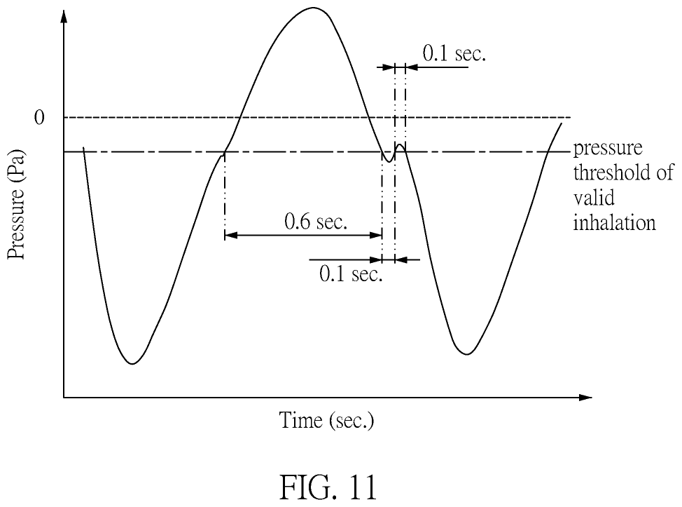

As shown in the fifth aspect of FIG. 11, based on a premise that the user's first inhalation is determined as a valid inhalation, since a duration of the user's inhalation (i.e., a period of time during which a pressure is measured to be lower than the pressure threshold) is greater than 0.25 seconds, the function of the control module 14 driving the atomization module will be invalidated when next time a valid inhalation is detected. Subsequently, since a stop time (i.e., a period of time during which the pressure measured to be greater the pressure threshold) after the user stops inhaling is 0.6 seconds, which is greater than 0.3 seconds, when the user enters a second inhalation, the invalidation of the function of the control module 14 driving the atomization module 10 will be deactivated, then the atomization module 10 performs atomization and dosing. Then, since a duration of the user's second inhalation is 0.1 seconds, which is less than 0.25 seconds, the function of the control module 14 driving the atomization module 10 will not be invalidated. Therefore, even if a second stop time is 0.2 seconds, which is less than 0.3 seconds, the atomization module 10 will still atomize the medicinal liquid when the user is in the third inhalation.

Through the above filtering standard, misjudgments caused by excessive irregular short breathing can be effectively eliminated, thereby avoiding invalid triggering.

It should be noted that although the aforementioned aspects of FIGS. 7 to 11 compare the duration corresponding to the pressure change during breathing with the locking time threshold and the unlocking time threshold, the present disclosure is not limited thereto. In other words, the present disclosure can also compare the duration corresponding to changes of air flow during breathing with the locking time threshold and the unlocking time threshold, so as to provide a mechanism for filtering multiple invalid triggers.

### BENEFICIAL EFFECTS OF THE EMBODIMENTS

In conclusion, in the atomization device and the method for predicting the atomization time provided by the present disclosure, breath prediction and determination can be performed based on the user's individual breathing time, thereby automatically adjusting the dosing time without being affected by differences in individual breathing rates. In addition, the atomized medicinal liquid can be absorbed by the user more efficiently, avoiding waste when exhaling.

On the other hand, in the atomization device and the method for predicting the atomization time provided by the present disclosure, a mechanism is further provided to avoid false triggering and filter multiple invalid triggers, and can also provide a corresponding feedback mechanism (such as vibration) based on operating status to allow relevant determinations to be made by the user.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A method of predicting atomization time adapted to an atomization device, the atomization device including a control module, an atomization module and a breathing sensing module, and the method comprising:
configuring the breath sensing module to detect one or more inhalations of a user using the atomization device, so as to generate one or more records of initial breath data correspondingly; and
configuring the control module to:
compare the one or more records of inhalation data in the records of initial breath data with a valid inhalation standard to obtain one or more records of valid inhalation data;
statistically analyze the one or more records of the valid inhalation data to generate a predicted value of inhalation time;
calculate an atomization time according to the predicted value of the inhalation time; and
generate a driving signal to drive the atomization module to perform atomization according to the atomization time.

2. The method according to claim 1, wherein the breath sensing module includes at least one of a pressure sensor and a flow sensor, and each of the one or more records of the inhalation data includes at least one of pressure data, flow data and duration corresponding to each inhalation of the user.

3. The method according to claim 2, wherein the valid inhalation standard includes at least one of a pressure threshold, a flow threshold, and a time threshold, and the process of comparing the one or more records of the inhalation data with the valid inhalation standard to obtain the one or more records of the valid inhalation data includes:
executing a determination process for each record of the inhalation data, wherein, each record of the inhalation data is determined to be one of the records of the valid inhalation data when the corresponding pressure data is lower than the pressure threshold or the corresponding flow data is higher than the flow threshold, and the corresponding duration is greater than the time threshold.

4. The method according to claim 2, wherein the valid inhalation standard includes at least one of a pressure threshold, a flow threshold and a time determination value, and the process of comparing the one or more records of the inhalation data with the valid inhalation standard to obtain the one or more records of the valid inhalation data includes:
executing a determination process for each record of the inhalation data, wherein, each record of the inhalation data is determined to be one of the records of the valid inhalation data when the corresponding pressure data is lower than the pressure threshold or the corresponding flow data is higher than the flow threshold, and the corresponding duration is greater than the time determination value;
wherein the time determination value is obtained by multiplying the predicted value of the inhalation time by a user ratio associated with the user.

5. The method according to claim 2, wherein the process of statistically analyzing the one or more records of the valid inhalation data to generate the predicted value of the inhalation time further includes:
obtaining N records of the duration corresponding to last N records of the one or more records of the valid inhalation data; and
calculating the predicted value of the inhalation time according to the obtained N records of the duration, wherein N is a positive integer greater than or equal to 2.

6. The method according to claim 5, wherein, when a record quantity of the one or more records of the valid inhalation data is less than N, durations of vacant records of the valid inhalation data relative to N are replaced by a predetermined time value and used to calculate the predicted value of the inhalation time.

7. The method according to claim 5, wherein the process of calculating the predicted value of the inhalation time according to the obtained N records of the duration further includes:
calculating an average of the obtained N records of the duration to serve as the predicted value of the inhalation time.

8. The method according to claim 2, wherein the process of comparing the one or more records of the inhalation data with the valid inhalation standard to obtain the one or more records of the valid inhalation data further includes:
in response to the durations corresponding to the M consecutive records of the inhalation data being less than a time standard of the valid inhalation standard, replacing a last one of the consecutive M records of the inhalation data with one record of the valid inhalation data,
wherein M is a positive integer greater than or equal to 2.

9. The method according to claim 3, wherein the control module includes a filtering standard having a locking time threshold and an unlocking time threshold, and the method further comprises: configuring the control module to: after the one or more records of the inhalation data are determined to be the valid inhalation data and in response to the duration corresponding to a current inhalation of the user being greater than the locking time threshold, activate an invalidation of a function triggered by the control module sending a drive signal to drive the atomization module when one of the inhalations contiguous to the current inhalation of the user is detected to be a valid inhalation; and when a stop time that is continually accumulated after the user stops inhaling is greater than the unlocking time threshold, deactivate the invalidation of the function triggered by the control module sending the drive signal.

10. The method according to claim 1, wherein the atomization time is the predicted value of the inhalation time multiplied by a predetermined ratio, and the atomization time does not exceed an upper limit of a dosing time.

11. The method according to claim 1, wherein the atomization device has a standby time, and when a valid inhalation is not detected for more than the standby time after the atomization device is started, the atomization device returns to a locked state.

12. An atomization device, comprising:
an atomization module;
a breath sensing module configured to detect one or more inhalations of a user using the atomizing device, so as to generate one or more records of initial breath data correspondingly; and
a control module electrically connected to the atomization module and the breath sensing module, wherein the control module is configured to:
compare the one or more records of inhalation data in the records of initial breath data with a valid inhalation standard to obtain one or more records of valid inhalation data;
statistically analyze the one or more records of the valid inhalation data to generate a predicted value of inhalation time;
calculate an atomization time according to the predicted value of the inhalation time; and
generate a driving signal to drive the atomization module to atomize a to-be-atomized medicine according to the atomization time.

13. The atomization device according to claim 12, wherein the breath sensing module includes at least one of a pressure sensor and a flow sensor, and each of the one or more records of the inhalation data includes at least one of pressure data, flow data and duration corresponding to each inhalation of the user.

14. The atomization device according to claim 13, wherein the valid inhalation standard includes at least one of a pressure threshold, a flow threshold, and a time threshold, and the process of comparing the one or more records of the inhalation data with the valid inhalation standard to obtain the one or more records of the valid inhalation data includes:
executing a determination process for each record of the inhalation data, wherein, each record of the inhalation data is determined to be one of the records of the valid inhalation data when the corresponding pressure data is lower than the pressure threshold or the corresponding flow data is higher than the flow threshold, and the corresponding duration is greater than the time threshold.

15. The atomization device according to claim 13, wherein the valid inhalation standard includes at least one of a pressure threshold, a flow threshold and a time determination value, and the process of comparing the one or more records of the inhalation data with the valid inhalation standard to obtain the one or more records of the valid inhalation data includes:
executing a determination process for each record of the inhalation data, wherein, each record of the inhalation data is determined to be one of the records of the valid inhalation data when the corresponding pressure data is lower than the pressure threshold or the corresponding flow data is higher than the flow threshold, and the corresponding duration is greater than the time determination value;
wherein the time determination value is obtained by multiplying the predicted value of the inhalation time by a user ratio associated with the user.

16. The atomization device according to claim 13, wherein the process of statistically analyzing the one or more records of the valid inhalation data to generate the predicted value of the inhalation time further includes:
obtaining N records of the duration corresponding to last N records of the one or more records of the valid inhalation data of the one or more records of the valid inhalation data; and
calculating the predicted value of the inhalation time according to the obtained N records of the duration, wherein N is a positive integer greater than or equal to 2.

17. The atomization device according to claim 16, wherein, when a record quantity of the one or more records of the valid inhalation data is less than N, durations of vacant records of the valid inhalation data relative to N are replaced by a predetermined time value and used to calculate the predicted value of the inhalation time.

18. The atomization device according to claim 16, wherein the process of calculating the predicted value of the inhalation time according to the obtained N records of the duration further includes:
calculating an average of the obtained N records of the duration to serve as the predicted value of the inhalation time.

19. The atomization device according to claim 13, wherein the process of comparing the one or more records of the inhalation data with the valid inhalation standard to obtain the one or more records of the valid inhalation data further includes:
in response to the durations corresponding to the M consecutive records of the inhalation data being less than a time standard of the valid inhalation standard, replacing a last one of the consecutive M records of the inhalation data with one record of the valid inhalation data,
wherein M is a positive integer greater than or equal to 2.

20. The atomization device according to claim 14, wherein the control module includes a filtering standard having a locking time threshold and an unlocking time threshold, and the control module is further configured to: after the one or more records of the inhalation data are determined to be the valid inhalation data and in response to the duration corresponding to a current inhalation of the user being greater than the locking time threshold, activate an invalidation of a function of the control module sending a drive signal to drive the atomization module when one of the inhalations contiguous to the current inhalation of the user is detected to be a valid inhalation; and when a stop time that is continually accumulated after the user stops inhaling is greater than the unlocking time threshold, deactivating the invalidation of the function of the control module sending the drive signal.

21. The atomization device according to claim 12, wherein the atomization time is the predicted value of the inhalation time multiplied by a predetermined ratio, and the atomization time does not exceed an upper limit of a dosing time.

22. The atomization device according to claim 12, wherein, when a valid inhalation is not detected for more than the standby time after the atomization device is started, the atomization device returns to a locked state.
